(19) United States
(12) Patent Application Publication
(10) Pub. No.: US 2003/0060813 A1
(43) Pub. Date: Mar. 27, 2003

(54) Devices and methods for safely shrinking tissues surrounding a duct, hollow organ or body cavity

(76) Inventors: Marvin P. Loeb, Huntington Beach, CA (US); L. Dean Crawford, Irvine, CA (US); Randy P. Graham, Irvine, CA (US); Mark A. Siminuk, Lake Forest, CA (US)

Correspondence Address: IP FOCUS LAW GROUP, LTD, 608 NORTH CARLYLE LANE, SUITE 100, ARLINGTON HEIGHTS, IL, 60004, US

(21) Appl. No.: 09/961,692

(22) Filed: Sep. 22, 2001

(51) Int. Cl. A61B 18/24

(52) U.S. Cl. 606/17; 606/10; 606/13

(57) ABSTRACT

A medical device applies radiant energy to tissue surrounding or underlying the surface of a duct, hollow organ or body cavity. The energy is emitted through an expandable, energy-transmissive balloon in which a fluid coolant is circulated to cool the surface of the duct, hollow organ or body cavity and the tissue immediately underlying the surface of the duct, hollow organ or body cavity. The device includes an elongated transmission line extending through a catheter, having a proximal end portion, which is connectable to a source of radiant energy, and a distal end portion, to which a radiant energy emitter is coupled. The balloon is mounted on the distal end of the catheter and extends over the emitter. The catheter contains an inlet confined fluid passageway and an outlet confined fluid passageway to provide fluid coolant circulation through the balloon. Microscopic albumen microspheres or particles of quartz or silica are suspended in the fluid coolant to more uniformly diffuse the radiant energy.

### FIELD OF THE INVENTION

This invention relates to medical devices and procedures for applying radiation to mammalian tissue underlying or surrounding a duct, hollow organ, or body cavity, particularly for purposes such as shrinking or coagulating of the tissue.

### BACKGROUND OF THE INVENTION

Devices have been developed to treat gastroesophageal reflux disease (GERD), fecal incontinence and benign prostatic hyperplasia (BPH) by inserting electrodes emitting radiofrequency (RF) energy into the tissue surrounding the esophagus in the area of the sphincter, into the tissue surrounding the anus or into the enlarged lobes of the prostate, respectively to cause coagulation, shrinkage or scarring thereof. In addition to causing bleeding, the electrodes become very hot and can damage the endothelial lining of the esophagus, anus and urethra, causing pain and other adverse effects. Also, RF energy can cause erratic tissue effects.

Laser energy has been used to coagulate the lobes of the prostate by positioning a side-firing, fiber optic device opposite thereto. While the lobes of the prostate can be easily coagulated, the urethra in the lased area and the immediately underlying tissue, which provides the urethra's blood supply, is destroyed, resulting in pain, dysuria and irritative symptoms which can persist for weeks.

Staples, slings and various other surgical appliances have been developed to treat female stress incontinence (FSI) by lifting or supporting the vaginal floor, but these require surgery and entail the risk of infections and poor results. Injections of bovine collagen to provide bulk to the tissues surrounding the urethra and support the vagina offer a temporary benefit, but do not provide long term relief, as the injected collagen is absorbed and migrates away from the injection site in six months or so.

When the annulus of the heart's mitral valve becomes loose and the cusps (leaflets) of the valve fail to close, blood is regurgitated out of the mitral valve, instead of exiting the left ventricle through the aortic valve into the aorta, which over time results in congestive heart failure and other complications. Surgical repair or replacement of the mitral valve entails substantial morbidity, mortality and cost.

In cosmetic and dermatologic procedures, laser energy is applied to the skin to cause shrinkage and growth of the new collagen in tissues lying beneath the epidermis to treat wrinkles or to coagulate unattractive blood vessels beneath the skin. To prevent thermal damage to the epidermis, a coolant, such as a cryogenic gas, ambient air, a fluid spray, a refrigerated, transparent gel or a cooled fluid circulated through a container with quartz or fused silica windows, can be applied to the surface of the skin to cool the epidermis. However, none of these techniques is feasible within the body, such as in a duct, hollow organ or body cavity, as contemplated hereby.

It would be desirable to provide long lasting relief to persons suffering from conditions such as GERD, fecal incontinence, FSI, BPH, vesico-uretal reflux (VUR), incompetent heart valves, and other conditions, without having to puncture or cut tissue, eliminating bleeding and reducing the risk of infections and other complications, in a minimally invasive procedure, with minimal morbidity and cost.

### SUMMARY OF THE INVENTION

The present invention is embodied in a medical device and related method for radiant-energy treatment of mammalian tissue surrounding or underlying a duct, hollow organ or body cavity with simultaneous tissue cooling. The method enables the radiant energy to cause scarring, shrinkage and coagulation of the tissue surrounding or underlying the duct, hollow organ or body cavity, while reducing damage to untargeted tissue areas such as the duct or endothelial lining of the hollow organ or body cavity, as well as the immediately underlying tissue, which provides its blood supply. In accordance with the apparatus and method aspects of the invention, radiant energy transmitted along a catheter based transmission line may be directed at tissue through a radiant energy emitter that is substantially surrounded by a circulating balloon-enclosed fluid coolant.

The apparatus aspect of this invention contemplates an elongated transmission line having a proximal end portion, which is connectable to a source of radiant energy, and a distal end portion. A radiant energy emitter is coupled to the distal end portion to receive energy therefrom. An associated insertion catheter defines a transmission line guideway for receiving the transmission line, as well as an inlet confined flow passageway and an outlet confined flow passageway to provide coolant circulation. Positioned to at least partially surround the emitter is an energy-transmissive balloon, sealed at the distal end region and in fluid communication with the inlet and outlet confined flow passageways for providing a tissue-contacting coolant chamber.

The invention relates generally to devices for applying light, microwave, ultrasound or other forms of electromagnetic energy through an energy transmissive, expandable balloon to induce heat in and shrink, by mechanically cross-linking collagen, coagulating and creating scarring in tissues surrounding a duct, hollow organ or body cavity, while simultaneously circulating a cooling fluid through the balloon to prevent damage to the sensitive endothelial lining and immediately underlying tissue of the duct, hollow organ or body cavity in contact with the balloon.

A method aspect of this invention contemplates the positioning of an expandable coolant balloon carried by the distal end of an energy-emitting catheter adjacent a tissue to be treated. The catheter includes a radiant energy emitter at least partially surrounded by the balloon. Thereafter, one method aspect contemplates first circulating fluid coolant through the expandable coolant balloon to expand the coolant balloon and cool the tissue in contact with the coolant balloon, and then energizing the emitter at a predetermined power level to emit radiant energy so as to irradiate the tissue to produce a zone of shrinkage, coagulation or scar tissue in the irradiated tissue, while continuing to circulate fluid coolant through the balloon to preserve the integrity of the duct or endothelial lining of the hollow organ or body cavity.

In an alternative method, the steps of circulating the cooling fluid and emitting radiant energy are initiated simultaneously.

In this manner tissue shrinkage, coagulation and scarring can be effectively performed within the body. The invention particularly applies to shrinking tissue surrounding the (a) esophagus in the area of the sphincter to treat gastroesophageal reflux disease (GERD), (b) the female urethra below the bladder to treat female stress incontinence (FSI), (c) the anus to treat fecal incontinence, (d) the vesico-uretal junction to treat vesico-uretal reflux (VUR), (e) the annulus of an incompetent heart valve or (f) the lobes of the prostate gland to treat benign prostatic hyperplasia (BPH).

An embodiment of the invention comprises a compliant or non-compliant, energy-transmissive balloon attached to the distal end of an elongate catheter. An emitter of light or other electromagnetic energy, including intense incoherent white or filtered light, coherent (laser) light, microwave or ultrasound energy or other electromagnetic energy, emitted laterally in a 90° arc or radially in a 360° arc from the axis of the catheter, is disposed at the distal end of the catheter, inside the balloon.

A fluid whose temperature is substantially lower than 37° C., such as tap water, water or saline cooled with ice, refrigerated water or saline or a cryogenic gas, is circulated through the balloon to cool the endothelial lining or inner surface of the duct, hollow organ or body cavity, while energy is being emitted. The emitted energy shrinks, coagulates and causes scarring in the tissue underlying the endothelial lining of the duct, hollow organ or body cavity, while the cooling fluid simultaneously counteracts the heat generated by the emitted energy in the endothelial lining of the duct, hollow organ or body cavity and the tissue immediately underlying the duct, hollow organ or body cavity, which provides the blood supply to the endothelium, substantially preventing damage thereto.

An alternate embodiment of the present invention is a laser treatment medical device including an elongate optical conduit having a proximal end region and a distal end region extending along a longitudinal axis and terminating in an energy delivery, distal end defined thereon for emitting laser radiation transmitted by the conduit. The proximal end region is adapted for connection to a laser energy source. A beam splitting lateral emitter is mounted on the distal end region of the conduit and is operably associated with the distal end for directing laser energy to the animal tissue. An insertion catheter defines an inlet confined flow passageway, an outlet confined flow passageway, and a guideway for receiving the optical conduit. A laser-energy-transmissive balloon at least partially surrounding the emitter is mounted at the distal end of the catheter for providing a tissue-contacting coolant chamber. The chamber is in fluid communication with the inlet and outlet confined flow passageways.

In a preferred embodiment, the lateral emitter includes an element defining a cavity within which the distal end of the optical conduit is received. The cavity has a distal end wall for blocking transmission of a laser energy beam coaxial with the distal end of the conduit. The emitter also includes a laterally open aperture to the cavity that is also open for fluid communication from outside the element through the aperture into the cavity, and a beam splitter, disposed entirely within the cavity, for receiving laser energy transmitted and for directing at least a first portion of the laser energy along a lateral beam path through the aperture. The lateral emitter optionally includes an extension and an atraumatic tip movably secured to the extension such that the balloon has a first waist sealed to the catheter and a second waist sealed to the atraumatic cap.

In a more preferred design, the beam splitter has a reflecting surface defined on the closed distal end wall arranged to reflect a first portion of the received laser energy.

Numerous other advantages and features of the present invention will be readily apparent to those skilled in the art from the following detailed description of the preferred embodiment of the invention, the drawings, and the appended claims.

### BRIEF DESCRIPTION OF THE DRAWINGS

In the accompanying drawings that form part of the specification,

FIG. 1 is a schematic view of a medical device according to the present invention;

FIG. 2 is an enlarged cross-sectional view of a laser based medical device according to the present invention;

FIG. 3 is a sectional view taken along the plane 3-3 in FIG. 2;

FIG. 4 is a sectional view showing an alternate embodiment of the passageway details shown in FIG. 3;

FIG. 5 is an enlarged fragmentary sectional view of the distal end portion of a medical device according to an alternate embodiment of the invention;

FIG. 6 is an enlarged fragmentary sectional view of the distal end portion of a medical device according to an embodiment of the invention in which an inner interface chamber is provided about the emitter;

FIG. 7 is an enlarged fragmentary sectional view of the distal end portion of a medical device according to another alternate embodiment of this invention;

FIG. 8 is an enlarged fragmentary sectional view of the distal end portion of a medical device according to yet another alternate embodiment of this invention;

FIG. 9 is an enlarged fragmentary sectional view of the distal end portion of a medical device according to yet another alternate embodiment of this invention;

FIG. 10 is a partial, external view of the end portion of the medical device of the present invention positioned in a female urethra;

FIG. 11 is a partial, external view of the end portion of the medical device of the present invention positioned in an esophagus in the area of the sphincter;

FIG. 12 is a partial, external view of a medical device according to the present invention, positioned in the mitral valve;

FIG. 13 is a different partial, external view of a medical device according to the present invention, positioned in the mitral valve;

FIG. 14 is a partial, external view of a device according to the present invention, positioned through the male urethra and within the prostate;

FIG. 15 is a partial, external view of a medical device according to the present invention, positioned in the anus; and

FIG. 16 is a partial, external view of an alternate embodiment of the distal end of a device according the present invention including a protective shield substantially surrounding the coolant balloon.

In the FIGURES, a single block or cell may indicate several components that collectively perform the identified function. Likewise, a single line may represent several individual signals.

### DESCRIPTION OF THE PREFERRED EMBODIMENTS

The invention disclosed herein is, of course, susceptible of embodiment in many different forms. Shown in the drawings and described herein below in detail are preferred embodiments of the invention. It is to be understood, however, that the present disclosure is an exemplification of the principles of the invention and does not limit the invention to the illustrated embodiments.

FIG. 1 depicts a general embodiment of the present invention, system 10. System 10 includes a source of light or other electromagnetic energy 11, such as a source of intense white or filtered incoherent light, coherent (laser) light, microwave or ultrasound energy, coupled to transmission line (or conduit) 12, such as an optical fiber, to transfer incoherent or coherent light energy, or a bound pair of insulated wires to transfer electrical energy. Transmission line 12 extends from energy source 11, through a liquid seal in the form of a gasket 13, into a flexible or rigid elongate catheter 14, whose distal end 15 is rounded or blunt ended to be relatively atraumatic to tissue, and terminates within balloon 16, which is disposed about the distal end portion of catheter 14.

All suitable balloon constructions result in a balloon that is relatively transparent to and substantially undamaged by the light or other electromagnetic energy to be transmitted therethrough. As used herein, the terms “energy transmissive,” “transparent,” “laser transmissive” and the like are a reference to the capability of the balloon (or balloon walls) to allow the transmission of radiant energy when the balloon is expanded. It is recognized that when collapsed, and therefore, possibly interfolded, the balloon may be damaged by or less capable or tolerant of radiant energy transmission therethrough.

More generally, the balloon is constructed of a material which can transmit irradiation without being substantially damaged. That is, there can be some slight damage to the balloon by the irradiation, but the integrity of the overall structural integrity of the balloon is maintained. The type of material used to construct the balloon can vary depending on the type of radiant energy used. If the balloon is only partially transparent to the radiant energy used, the balloon material should preferably have a relatively high melting temperature.

Furthermore, the balloons may be substantially compliant or substantially non-compliant, selected according to the condition to be treated. The relative elasticity of the coolant balloon will vary according to the material of construction, which is preferably polymeric, and the related material processing. Preferred materials include a natural rubber, a polyurethane, a polyethylene, a polyethylene terephthalate, a polyester, a copolyester, a polyvinyl chloride, a copolymer of vinyl chloride and vinylidene chloride, and composites thereof.

Preferred for preparing a substantially non-compliant balloon are a polyethylene terephthalate (PET), a polyester, a copolyester and a polyurethane. The type of material used to construct the balloon can vary depending on the type of radiant energy to be used to treat the tissue. A preferred material for preparing a relatively compliant balloon is a copolymer of vinyl chloride and vinylidene chloride commercially available under the tradmark SARAN from Dow Chemical Company (Midland, Mich.). A SARAN balloon can be manufactured in a sack-like configuration with any seams located toward the inside of the balloon. A SARAN balloon can be used with an Argon, KTP, diode or Nd:YAG laser.

Another material suitable for preparing a more compliant balloon is a substantially clear natural rubber. A natural rubber balloon can also be used with an Nd:YAG laser. A substantially transparent, natural rubber balloon material is prepared by curing latex on form in the desired shape of the balloon. The balloon can then be sealingly mounted on the distal region of the catheter.

The thickness of the balloon can vary according to the selected type fluid coolant and the type of radiant energy to be utilized. The thickness and tensile strength of the balloon is such that it can withstand the inflated pressure of the non-compressible fluid when fully inflated. This pressure is typically about 2 to 4 atmospheres of pressure, although the pressure level can be greater or lesser.

Returning to FIG. 1, male luer connector 17 is affixed to and is in fluid communication with an inlet confined flow channel passageway (not separately shown) within catheter 14, to which female luer connector 18 may be removably attached. A first end of inflow tube 19 is affixed to female luer connector 18, and its opposite end is affixed to female luer connector 20, which is removably attached to male luer connector 21. Male luer connector 21 is affixed to and is in fluid communication with the coolant supply outlet of a circulator in the form of a pump 22.

A preferred coolant fluid is selected from the group consisting of chilled de-ionized water, chilled saline, and cryogenic-state gas. In operation, the cooling fluid is cooled to a temperature substantially lower than 37° C.

A preferred circulator is selected from the group consisting a peristaltic pump, a diaphragm pump, a piston pump, a bellows pump, a syringe pump, a roller and ball pump.

Pump 22 is in fluid communication with a cooler 23, which may consist of a refrigeration unit, utilizing a compressed gas such as Freon, wafers of a dielectric material or other cooling mechanism.

Pump 22 has a controller (not separately shown) and a display 24 containing “up” and “down” icons, which enable the operator to call for the pump to deliver fluid at a desired pressure, as shown, such as about 0.1 to 2.5 pounds per square inch, or a desired volume of fluid, such as about 2 to 30 ml per minute (not separately shown). Cooler 23 has a controller (not separately shown) and a display 25 containing “up” and “down” icons, enabling the operator to call for the cooler to deliver fluid at a desired temperature, preferably about 3° C. to 33° C., depending on the type, amount and duration of energy being applied.

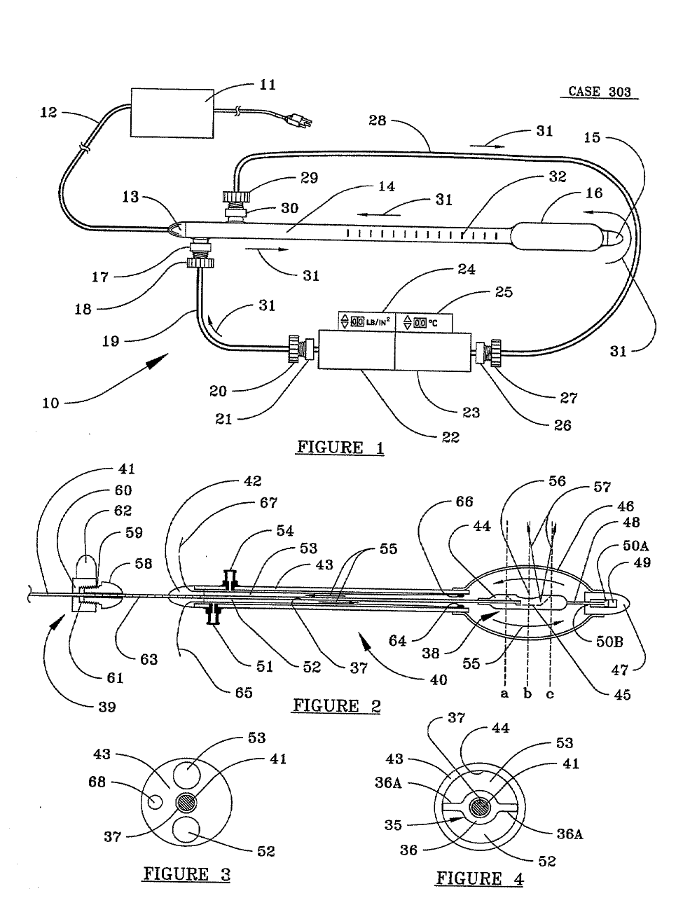

The output of pump 22 is preferably controlled based on a measurement from a pressure transducer or sensor (FIG. 2) in the fluid pathway, and the level of cooling provided by cooler 23 is preferably controlled based on a thermocouple measurement or other temperature sensor (FIG. 2) in the fluid pathway. The pressure transducer and thermocouple are mounted within pump 22 or cooler 23, within balloon 16 or catheter 14, or elsewhere within device 10, with electrical connections (not separately shown) to the pump controller and cooler controller, respectively.

Other display and control mechanisms are contemplated. For example, measurements of fluid coolant pressure and fluid coolant temperature are used in combination by a controller to reset the output of both pump 22 and cooler 23.

Taken together, pump 22 and cooler 23 form a cooling system. As illustrated, circulator pump 22 and cooler 23 are combined into a single module.

In an alternate embodiment, instead of pump 22, inflow of tap water to device 10 is controlled by a control valve, a controller linked to the valve and a display system, with return fluid diverted to a drain. In an alternate embodiment, pump 22 is configured to draw fluid from a reservoir, for example, a container of water or saline and ice, returning the fluid to the reservoir or diverting it to a drain.

Male luer connector 26 serves as a fluid coolant return and is affixed to and is in fluid communication with cooler 23. Female luer connector 27, which may be removably attached to male luer connector 26, is affixed to a first end of outflow tube 28, and its opposite end is affixed to female luer connector 29, which is removably attached to male luer connector 30, which is affixed to and in fluid communication with an outlet confined flow passageway defined in catheter 14. Female luer connectors 17 and 30 are each in fluid communication with a outlet confined flow passageway defined in catheter 14, which enable the fluid to flow in the direction shown by arrows 31, as well as into and out of balloon 16, as also shown in FIGS. 2 and 5 through 9 and described below.

Optionally, catheter 14 has scaled position markings 32 around its shaft, for example at about 0.5 cm intervals or other convenient intervals, enabling the operator to determine the distance balloon 16 mounted on catheter 14 has been inserted into a duct, hollow organ or body cavity from its entry point, the operator having earlier determined the desired depth by endoscopy, ultrasound imaging, fluoroscopy or other means.

For the emission of microwave energy, the source of radiant energy is a microwave generator, the transmission line is preferably a coaxial conductive cable, and the emitter is a microwave antenna. For the emission of ultrasonic energy, the emitter is preferably a piezoelectric acoustic emitter.

In FIG. 2, medical device 40 is shown. Device 40 comprises a transmission line in the form of fiber optic conduit (or cable) 41, which in this embodiment is a quartz or fused silica optical fiber, whose outer surface is covered by a glass cladding (not separately shown), which is in turn covered by a vinyl cladding (not separately shown) which is in turn covered by a “buffer” coating (not separately shown). Conduit 41 is slidably received in guideway 37 defined in an insertion catheter 43 through a fluid tight gasket 42. The gasket is attached to the proximal end of catheter 43 by an adhesive, thermal bonding or the like. The distal end of conduit 41 terminates in a radiant emitter subassembly 38. More specifically, the distal end of conduit 41 is encased within metal tip 44, which is fixed to conduit 41 by crimping, adhesive or both.

Metal tip 44 has a cavity formed within its body, as indicated by dotted line 45. The distal face of the cavity, opposite the distal end of conduit 41, is slanted at an angle from the axis of conduit 41, preferably at an angle of about 45°. The slanted face of metal tip 44 may be plated, coated or overlaid with a material reflective to the wavelength of light being utilized, such as gold, silver, copper, platinum or a dielectric coating. The design of a suitable metal tip 44 and its reflective material is more fully described in co-owned U.S. Pat. No. 5,649,924 to Everett et al., the disclosure of which is incorporated herein by reference to the extent it is not inconsistent.

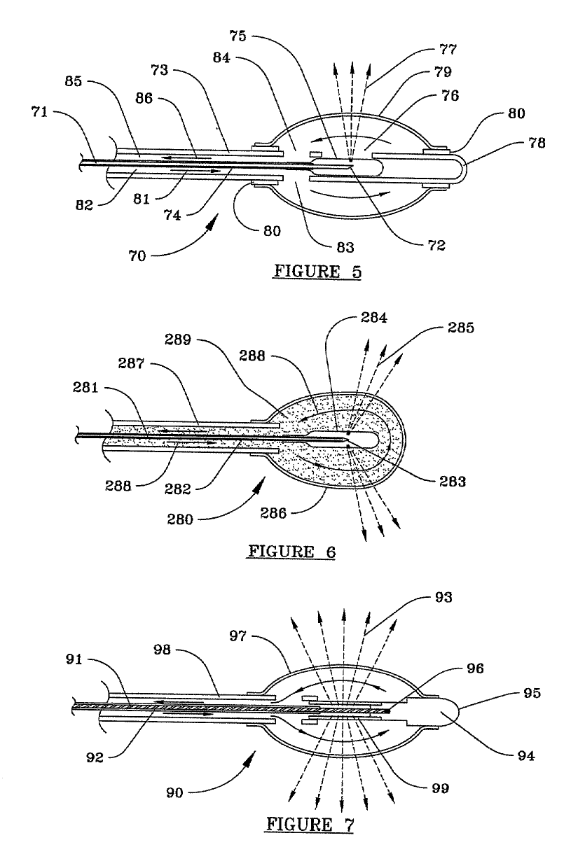

Fluid enters female luer connector 51, which is in fluid communication with confined flow passageway 52 (or channel) defined in catheter 43. Fluid passageway 52 may be created as shown in catheter cross-sectional FIG. 3. FIG. 4 is a cross-section view of an alternate catheter configuration for providing the confined flow passageways 52 and conduit guideway 37.

Fluid entering channel 52 fills and expands coolant balloon 46, whose proximal end is attached by an adhesive or thermal bonding to the exterior of catheter 43, at or near its distal end, and whose distal end is similarly attached to the exterior of an atraumatic, rounded or blunt ended ceramic or plastic tip 47.

An extension 48 of metal tip 44 is slidably disposed within slide 49 in plug 47. Extension 48 has outwardly extending flange 50A at its distal end. Inwardly extending flange 50B, at the proximal end of slide 49 in plug 47, prevents outward extending flange 50A and extension 48 from inadvertently being withdrawn out of slide 49.

Coolant leaves balloon 46 through an outlet confined flow passageway (or channel) 53 defined in catheter 43 and exits through male luer connector 54. Fluid passageway 53 and conduit guideway 37 can be created as shown in catheter cross-sectional FIGS. 3 and 4. Arrows labeled with reference number 55 show the direction of fluid flow into and out of catheter 43 and balloon 46.

In an alternate an embodiment, outflow fluid passageway 53 can be eliminated and a relief valve, which opens at a pre-set pressure, may be mounted through balloon 46 to allow coolant to exit balloon 46 into the duct, hollow organ or body cavity when a pressure level sufficient to properly expand balloon 46 is reached. In this device, the cooling fluid must be physiologically compatible, such as sterile water or saline.

In operation, light energy emitted from the distal end of conduit 41 strikes the distal 45° slanted, reflective surface of cavity 45 of metal tip 44 and exits through port 56 laterally from the axis of conduit 41, in the direction shown by arrows 57.

To facilitate the handling of conduit 41 within guideway 37 of catheter 43, device 40 includes a clampable handle subassembly 39 slidably received towards the proximal end region of conduit 41. In a preferred embodiment, the clampable operator handle takes the form of a compression fitting with a plastic or metal inner sleeve 58 moveably disposed over conduit 41, proximally from gasket 42 of catheter 43. Sleeve 58 has threads 59 about the exterior of its proximal end, as shown. Outer sleeve 60 is likewise moveably disposed over conduit 41 proximal to sleeve 58.

Outer sleeve 60 has threads 61 on its inner surface, which proximally become progressively of a smaller inside diameter. When outer sleeve 60 is screwed onto inner sleeve 58, progressively smaller threads 61 of outer sleeve 60 cause sleeve 58 to become removably fixed to conduit 41. When thereby clamped to conduit 41, the operator may easily grasp outer sleeve 60 to advance or withdraw conduit 41 within catheter 43.

Optionally, when energy is to be emitted, for example, in an arc of about 90°, laterally at an angle of about 90° from the axis of catheter 43, a wing, extension or protuberance 62, as shown, or a marking or other direction indicator on outer sleeve 60, when outer sleeve 60 is screwed onto inner sleeve 58 prior to insertion into the body, can be positioned in the direction in which light energy will exit light port 56. Thus, once inside the body, from the position of the wing or protuberance 62 (or marking on outer sleeve 60), the operator can ascertain the orientation of light port 56 of metal tip 44 within balloon 46 and the direction in which light energy will be emitted through balloon 46.

Conduit 41 has position markings 63 along at least a portion of its length, as shown, in intervals of, for example, 0.25 cm. Other marking intervals are contemplated. Thus, when conduit 41 is advanced into or withdrawn from catheter 43 through gasket 42, the operator can ascertain the longitudinal position of light port 56 within balloon 46.

For example, by rotating and advancing or withdrawing outer sleeve 60, once it has been fixed in place by compressing inner sleeve 58 on conduit 41, light energy may be emitted for selected periods of time, for example, such as at the 3, 6, 9 and at 12 o'clock positions at each of positions a, b and/or c, as shown by the dashed lines in FIG. 2, or in any other pattern of positions.

A preferred method of using a lateral-firing laser device is described in more detail in co-owned U.S. Pat. No. 5,437,660 to Johnson et al. which is incorporated herein by reference to the extent it is not inconsistent.

While FIG. 2 illustrates conduit 41 being moveably disposed within gasket 42 and catheter 43, an alternative embodiment has conduit 41 fixedly attached within gasket 42, and extension 48 fixedly attached within plug 47.

Preferably device 40 includes a temperature sensor in the form of a thermocouple 64 positioned towards the distal end of catheter 43 within channel 52. An insulated wire 65 extends from thermocouple 64 for use with a controller as described in reference to FIG. 1. Likewise, a pressure transducer 66 is preferably affixed towards the distal end region of catheter 43 within channel 53. An insulated wire 67 extends from transducer 66 to a suitable controller (not shown).

Thermocouple 64 and pressure transducer 66 described in reference to FIGS. 1 and 2 are alternatively positioned within the cooling system, i.e. the circulator pump and/or the cooler, or within the respective fluid inflow or outflow tubes, the catheter or the balloon, with insulated wires 65 and 67 extending therefrom through the tubes, catheter or gasket of such embodiments to their respective pump and cooler controllers.

FIG. 3 illustrates one embodiment of catheter 43 of device 40 in FIG. 2, which has been extruded with three longitudinal channels. Conduit 41 is movably disposed within central guideway or channel 37 of conduit 43. Inflow channel 52 at 6 o'clock and outflow channel 53 at 12 o'clock correspond to inflow and outflow channels 52 and 53, respectively, of catheter 43 of FIG. 2.

If the thermocouple 64 and pressure transducer 66 are affixed within channels 52 and 53, respectively, of catheter 43, as shown, wires 65 and 67, from thermocouple 64 and pressure transducer 67 extend through channels 52 and 53, respectively, of catheter 43, exit plug 42, as shown, and extend to their respective pump and cooler controllers. Optionally, a fourth channel 68 (FIG. 3) in catheter 43 provides a passageway for wires 65 and 67 to their respective pump and cooler controllers (not shown).

FIG. 4 illustrates another embodiment of catheter 43 of device 40 of FIG. 2. In this embodiment, catheter 43 defines a central bore 44 and includes a coextensive partition in the form of a hollow, vaned plastic insert 35 for dividing the central bore 44 of catheter 43 into conduit guideway 37, inlet passageway 52 and outlet passageway 53. More specifically, a hollow vaned plastic insert 35 consists of a central hollow tube 36 with, as shown, two vanes 36A extending from plastic insert 35 which extends from the proximal end face of gasket 42 of device 40 of FIG. 2, through catheter 43 and terminates at the distal end of catheter 43. Conduit 41 may be movably disposed within hollow tube 36. As shown, the two vanes 36A extend laterally from central hollow tube 36 of plastic insert 35 and form a friction, fluid seal against the inner wall of central bore 44 of catheter 43, creating inflow channel 52 and outflow channel 53.

Any number of vanes may be employed, such as three, four or more. Furthermore, either or both of channels 52 or 53 may enable insulated wires from a thermocouple or pressure transducer affixed at the distal end of catheter 43 or within balloon 46, to pass through catheter 43 and gasket 42 to their respective cooler and pump controllers.

FIG. 5 illustrates the distal end area for a medical device in accordance with an alternate embodiment of the present invention. Device 70 contains conduit 71, which in this embodiment is a quartz or fused silica optical fiber which is disposed within catheter 73. The distal end face 72 of conduit 71 is beveled at an angle from the axis of conduit 71, preferably at an angle of about 45°. Buffer coat and vinyl cladding 74 have been removed from the distal end portion of conduit 71. A quartz or fused silica capillary tube 75, whose distal end has been closed by fusing, and whose proximal end is attached to the bared portion of conduit 71 by thermal fusing or an adhesive, creates an air interface at the beveled distal end face 72 of conduit 71. An air interface is necessary for total internal reflection of light energy, which is seen to exit capillary tube 75 laterally at an angle of about 90° to about 110° from the axis of conduit 71, through light exit port 76 in catheter 73, as shown by arrows 77. In this embodiment, catheter 73 is preferably a metal tube or needle, preferably of stainless steel, whose distal end 78 has been closed and rounded into an atraumatic shape.

Balloon 79 is disposed over the distal end portion of catheter 73. To avoid metal catheter 73 becoming overheated during the emission of light energy and damaging balloon 79 at its points of attachment to catheter 73, optionally, plastic or ceramic sleeves 80 are attached, as shown, proximally and distally, respectively, from light exit port 80, about catheter 73 by an adhesive or the like, and the proximal and distal ends of balloon 79 may be respectively attached by an adhesive or the like to plastic or ceramic sleeves 80.

Arrow 81 indicates the direction of fluid inflow through channel 82 and port 83 of catheter 73 into balloon 79. Fluid flows out of balloon 79 through port 84 into channel 85 of catheter 73, in the direction of flow indicated by arrow 86.

FIG. 6 illustrates the distal end area for a medical device 280 in accordance with another alternate embodiment of the present invention. In this embodiment, conduit 281 is a quartz or fused silica optical fiber whose body is covered by a first glass cladding, a second vinyl cladding and a plastic buffer coating 282. The distal end face 283 of conduit 81 has been beveled into a cone shape, whose surface is inclined at an angle of about 45° from the axis of conduit 281.

Capillary tube 284 of quartz or fused silica, whose distal end has been closed by thermal fusing or melting, has been attached, as shown, over the bared distal end portion of conduit 281, by adhesive or thermal fusing. Capillary tube 284 creates an air environment or interface chamber at the cone shaped, distal end face 283 of conduit 281, which by total internal reflection causes the light energy to be emitted radially from the axis of conduit 281 in a 360° arc, as shown by arrows 285.

The proximal end of balloon 286 is attached to the distal end portion of plastic or metal catheter 287. Balloon 286 has been preformed, by molding or casting, to expand, when inflated, into a shape which extends beyond and does not contact the distal end of capillary tube 284. Fluid flow into and out of catheter 287 and balloon 286, as shown by arrows 288 is as described in reference to FIGS. 2, 3 and 4.

In a preferred embodiment, the cooling fluid contains a multiplicity of micro-particles 289 which reflect and diffuse the energy, but do not appreciably absorb the energy and heat the fluid, diminishing its cooling effect. While, as described in U.S. Pat. No. 4,612,938 to Dietrich et al. which is incorporated herein by reference, a liquid containing a multiplicity of tiny globules of fat, such as a commercially available high fat intravenous fluid, can reflect and diffuse the energy, the fat globules absorb some of the energy, heat the fluid and diminish its cooling effect.

A preferred diffusing coolant fluid contains a suspension of either a multiplicity of abumen microspheres, with a diameter of less than about 10 microns, preferably about 1 to about 4 microns or less, at a concentration of less than about 20 percent, preferably about 2 to about 15 percent, based on the total weight of the coolant. Most preferred is a multiplicity of inert, non-energy absorbing, microscopic quartz or fused silica particles, commonly referred to as fumed silica, such as the particles commercially available from Cabot Corporation (Boston, Mass.) under the trademark “Cab-O-Sil.” Preferred fumed silica particles have a diameter of less than about 50 microns, and more preferably, are in the range of about 0.2 to about 0.3 microns. Fumed silica particles are preferably present in the range of about 2.5 percent to 25 percent, and more preferably about 10 to 15 percent, based on the total weight of the coolant.

These preferred diffusing fluids described above can likewise be used in the devices described in reference to FIGS. 1, 2, 5 and 7 through 9.

FIG. 7 illustrates the distal end area for a medical device 90 in accordance with another alternate embodiment of the present invention. Medical device 90 includes a conduit 91, which in this embodiment is a quartz or fused silica optical fiber, from whose distal end both vinyl cladding and buffer coating 92 have been removed, as shown, enabling light energy to be emitted radially from the axis of bared conduit 91 in a 360° arc, as shown by arrows 93. In a preferred embodiment, the bared surface of conduit 91 has been sandblasted to further diffuse the emission of light energy therefrom.

The distal end of conduit 91 is fixedly attached, by an adhesive or the like, within plastic or ceramic plug 94, whose distal end 95 is rounded or blunt ended. Optionally, to reflect light energy back into conduit 91 and avoid overheating of plastic or ceramic plug 94, a coating or insert 96 of a reflective material, such as gold, silver, copper, platinum or a dielectric material, may be disposed over or opposite the distal end face of conduit 91. The proximal end of balloon 86 is attached by an adhesive or the like near the distal end of catheter 87. Fluid flow into and out of catheter 96 and balloon 97 is illustrated by the arrows, as described in reference to FIGS. 2 and 5.

The distal end of balloon 97 is attached to the exterior of plastic or ceramic plug 94 by an adhesive or the like. In this embodiment, quartz or fused silica tube 99 extends over and protects the bared end portion of conduit 91, which is fragile due to its buffer coat having been removed. The proximal end of tube 99 of quartz or fused silica is affixed within the distal end of catheter 97 and attached thereto by an adhesive or the like, as shown. The distal end of tube 99 is affixed to the proximal end of plug 94 by an adhesive or the like, as shown. However, if conduit 91 is an optical fiber of sufficient core diameter, such as 550 to 1000 microns, tube 99 may optionally be eliminated.

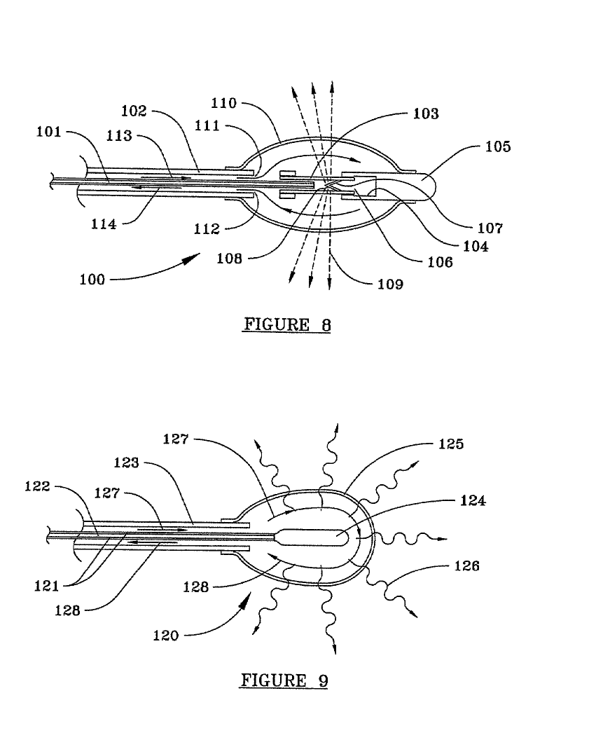

FIG. 8 shows the distal end portion of another embodiment of the present invention, medical device 100. Device 100 includes conduit 101, which in this embodiment is a quartz or fused silica optical fiber. Conduit 101 is fixedly disposed within metal or plastic catheter 102. The proximal end of quartz or fused silica sleeve 103 is attached within the distal end of catheter 101, by an adhesive or the like, and its distal end extends to and is fixedly attached by an adhesive or the like within recess 104 in rounded or blunt ended plastic or ceramic plug 105. As described in co-owned U.S. Pat. No. 5,242,438 to Saadatmanesh et al. which is incorporated herein by reference, metal fitting 106 is fixedly attached within recess 104 of plug 105 by an adhesive or the like. The distal end face 107 of metal fitting 106 is shaped into a cone, whose surface is preferably slanted at an angle of about 45° from the axis of conduit 101, as shown. Metal fitting 106 is made of a reflective material such as gold, silver, copper or platinum. Alternatively, as shown, a plating or insert 108 of a reflective material, such as gold, silver, copper, platinum or a dielectric material, as described heretofore, extends over or is affixed to distal end face 107 of metal fitting 106. Light energy emitted from the distal end of conduit 101 is reflected from reflective plating or insert 108 radially from the axis of conduit 101 in a 360° arc, as shown by arrows 109.

The proximal end of balloon 110 is attached near the distal end of catheter 102 by an adhesive or the like, and the distal end of balloon 110 is likewise attached to the exterior of plug 105. Fluid enters balloon 110 through inflow port 111 of catheter 102 and exits balloon 110 through outflow port 112 in catheter 102, in the respective directions shown by arrows 113 and 114.

The utilization of an alternate energy source is depicted in FIG. 9. In this embodiment, the distal end portion 120 of apparatus 10 of FIG. 1 includes a pair of insulated wires 121, which may optionally be attached to non-conductive strip or rod 122, and which extend through plastic or metal catheter 123 to an emitter 124 of microwave, ultrasound or other electromagnetic energy (not separately shown). About 10 to 300 watts, preferably about 30 to 200 watts, of microwave, ultrasound or other electromagnetic energy may be emitted radially through balloon 125 in a 360° arc, as shown by arrows 126. Alternatively the aforesaid energy is focused into a single beam or multiple beams of energy, which are emitted through balloon 125 and converge at a desired point in the tissue outside balloon 125.

The proximal end of balloon 125 is attached by an adhesive, thermal bonding or the like at or near the distal end of catheter 123. Balloon 125 has been preformed by molding or casting to expand, when inflated, into the shape shown, which extends beyond and does not contact energy source 124. Fluid flow in and out of catheter 123 and balloon 125 is indicated by arrows 127 and 128, respectively.

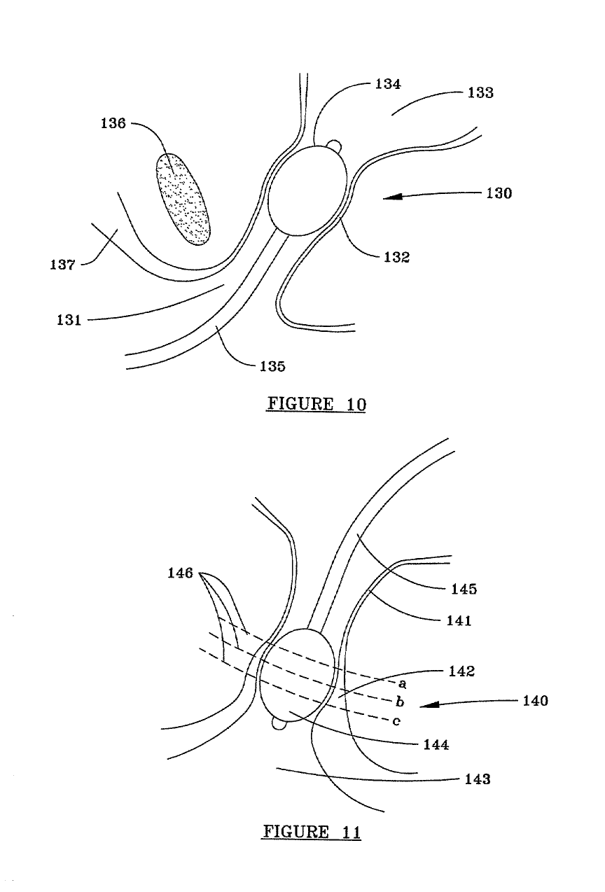

As seen in FIG. 10, a distal end portion 130 of apparatus 10 of FIG. 1 is shown inserted through vagina 131 into female urethra 132, to a desired position beneath bladder 133. Non-compliant or compliant balloon 134, which is attached to catheter 135, has been expanded, as shown, in urethra 132. The pubic bone 136 and pubis 137 are shown to the left.

To shrink, coagulate and cause scarring of the tissues surrounding the female urethra, to tighten the urethra and reduce or eliminate stress incontinence, 5 to 100 or more watts of white or filtered incoherent light, such as from a xenon lamp light source, coherent (laser) light, such as from an argon, KTP, diode, Nd:YAG or Holmium:YAG laser, or microwave or ultrasound energy is radially emitted through balloon 134 in a 360° arc, preferably by a device such as illustrated in FIGS. 6 through 9, for about 30 seconds to 10 minutes or longer, depending on the type and amount of energy utilized, while a cooling fluid (preferably water or saline in the case of argon, KTP, diode or Nd:YAG laser energy or carbon dioxide gas in the case of Holmium:YAG laser energy) is circulated through balloon 134. The coolant fluid is circulated through balloon 134 simultaneously with the emission of energy. However, it is most preferable to circulate the coolant fluid through balloon 134 before the emission of energy for a time period sufficient to cool the urethra and the tissue immediately lying therebeneath to a depth of about 1 to 5 mm, as well as circulating the cooling fluid during the emission of radiant energy.

While conventional optical fibers can be used with Argon, KTP, diode and Nd:YAG lasers, optical fibers with a low hydroxyl (OH) must be used with Holmium:YAG and similar lasers. If filtered light energy is desired, light of undesired wavelengths may be filtered out, leaving the desired wavelengths to be emitted. Desirable wavelengths include green or blue-green, which are well absorbed by and rapidly heat blood and, hence, the surrounding tissue.

Alternatively, 5 to 100 or more watts of incoherent or coherent light energy or focused microwave, ultrasound or other electromagnetic energy, as described above, may be emitted laterally, at an angle of about 90° to 110° from the axis of catheter 135, through balloon 134 in an arc of about 90°, preferably from a device such as illustrated in FIGS. 2, 5 or 9, for about 15 seconds to 5 minutes or longer, depending upon the type and amount of energy applied, at about 12 o'clock and about 6 o'clock (or at each of 5 and 7 and 11 and 1 o'clock) to avoid nerves and blood vessels which extend alongside the urethra at about 3 and 9 o'clock, or in any other desired pattern, with cooling fluid flowing as described above.

In FIG. 11, the distal end portion 140 of apparatus 10 of FIG. 1 is shown inserted into the esophagus 141 and positioned within the area of sphincter 142, above stomach 143. Compliant or non-compliant balloon 144, which is attached about the distal end of catheter 145, has been expanded within sphincter 142.

To shrink, coagulate and cause scarring in the tissue surrounding the esophagus in the area of the sphincter, to tighten the sphincter and reduce or eliminate GERD, preferably 5 to 100 or more watts of incoherent (white or filtered) or coherent (laser) light, microwave, ultrasound or other electromagnetic energy, as earlier described for FIG. 10, is radially emitted through balloon 144 in a 360° arc, for 30 seconds to 10 minutes or longer, depending on the type and amount of energy applied, while a cooling fluid has been earlier and is concomitantly or is simultaneously circulated through balloon 144 to cool the inner lining of esophagus 141. The above procedure may be repeated at one or more levels within the area of the sphincter, for example, at levels a, b and c, as shown by dotted lines 146.

Alternatively, as earlier described in reference to FIG. 10, 5 to 100 or more watts of light or focused ultrasound, microwave or other electromagnetic energy may be emitted laterally, at an angle of about 90° to 110° from the axis of catheter 145, in an arc of about 90° for 15 seconds to 5 minutes or longer, depending on the type and amount of energy used, for example, at each of the 3, 6, 9 and 12 o'clock positions at one or more of levels a, b, and c, or in any other desired pattern.

While coolant fluid can be circulated through the balloon during the emission of energy, preferably balloon 144 is positioned in the esophagus and inflated with coolant fluid for a sufficient period of time to pre-cool the endothelial lining of the esophagus and the tissue immediately underlying therebeneath, to a depth of about 1 to 5 mm, before and during the emission of energy.

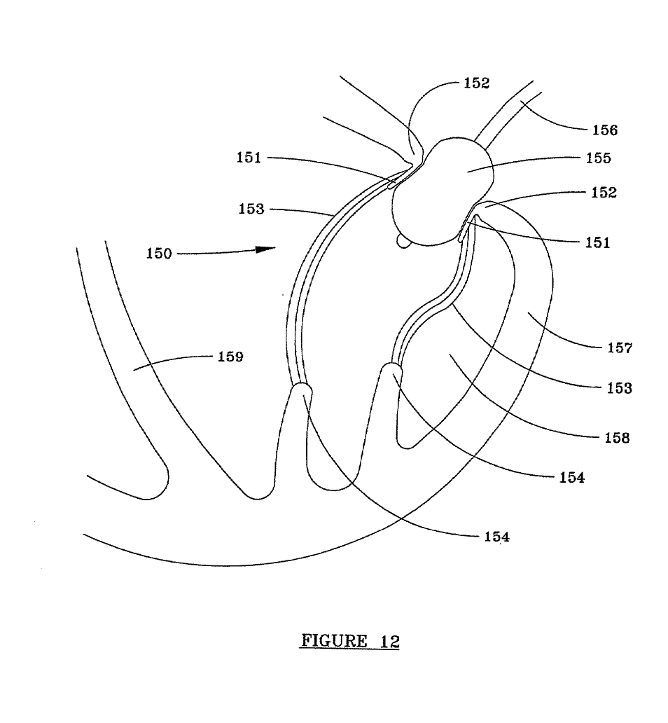

In FIG. 12, the distal end portion 150 of apparatus 10 of FIG. 1 is shown inserted between cusps (or leaflets) 151 of the mitral valve. Cusps 151 are attached to annulus 152 of the mitral valve, and are closed and opened by the chordae tendinae 153, when papillary muscles 154 contract and relax. If annulus 152 of the valve is loose, cusps 151 do not completely close, and blood leaks from the valve. Shrinking annulus 152 can cause cusps 151 to close properly and prevent blood from leaking (regurgitating) out of the mitral valve during the heart's compression.

Distal end 150 was inserted into the mitral valve from above cusps 151 (i.e. through the aorta), forcing cusps 151 inward. Balloon 155 is attached about the distal end of catheter 156. While a non-compliant material can be used to make balloon 155, preferably, as shown in this embodiment, balloon 155 is made of a compliant material and has been expanded within annulus 152. Wall 157 of left ventricle 158 and septum 159 are shown.

To shrink, coagulate and cause scarring in the tissue surrounding annulus 152 of the mitral valve, in order to tighten annulus 152 incoherent or coherent light, microwave, ultrasound or other electro-magnetic energy is emitted radially in a 360° arc through balloon 155 from a device, such as shown in FIG. 6, 7, 8 or 9, in the amounts and for the time periods described in reference to FIG. 10 above. A cooling fluid is circulated through balloon 155 either before the emission of energy, to pre-cool cusps 151 and annulus 152, and during the emission of energy to cool and prevent damage to the same, or solely during the emission of energy to cool and prevent damage as earlier described.

Alternatively, the incoherent or coherent light, focused ultrasound, microwave or other electromagnetic energy is emitted laterally, at an angle of about 90° from the axis of catheter 156 in an arc of about 90° to treat selected areas of annulus 152, such as at any or all of 3, 6, 9 and 12 o'clock, of the types, amounts and time periods described above. During or, preferably, before and during the emission of radiant energy, cooled fluid is circulated through balloon 155 to pre-cool the cusps and tissue immediately underlying the annulus.

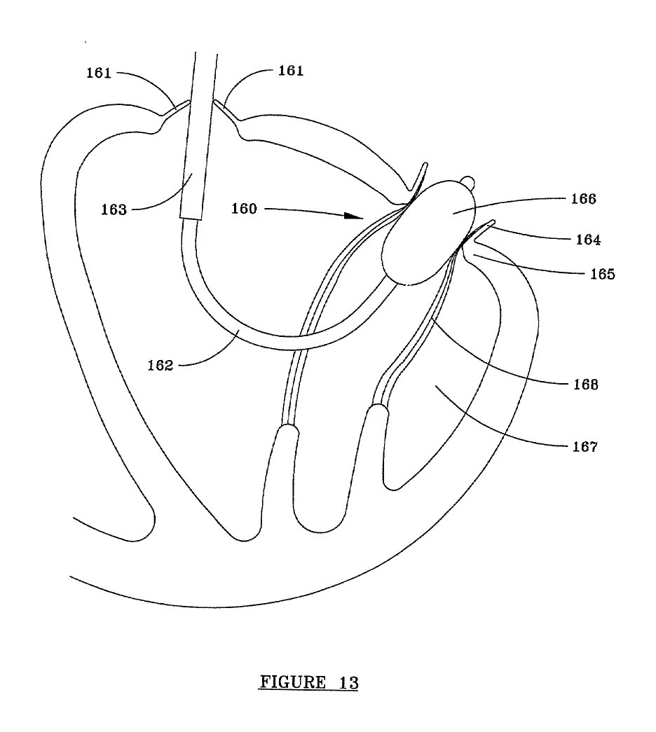

In FIG. 13, the distal end portion 160 of apparatus 10 of FIG. 1 is shown inserted through cusps 161 of the aortic valve. Catheter 162 may be introduced as shown, through cusps 161 of the aortic valve, by inserting it through an earlier positioned guiding catheter 163 as known in the art. Catheter 162 is articulated, for example by retracting wires extending through catheter 162 and attached at its distal end (not separately shown) or by incorporating a nitinol wire (not separately shown) within the body of catheter 162, which has been preformed into the curved shape shown. Catheter 162 has been inserted through cusps 164 of annulus 165 of the mitral valve from below, forcing cusps 164 outward. Balloon 166, attached about the distal end portion of catheter 162, can be made of a compliant material. However, in this method embodiment, balloon 166 is preferably made of a non-compliant material. Balloon 166 is attached about the distal end of catheter 162 by an adhesive or the like and has been expanded within annulus 165 of the mitral valve. The same energy sources, energy levels, patterns, positions, duration of emission and cooling fluid use as described for FIG. 10 are utilized for this method embodiment.

Visualization of catheter 162 and balloon 166 (if filled with a radio-opaque fluid) as well as left ventricle 167, cusps 164 and annulus 165 of the mitral valve may be provided by fluoroscopy. Preferably, an intra-cardiac ultrasound imaging device, such as the AcuNav® ultrasound catheter marketed by Acuson, Inc. of Mountain View, Calif., is inserted into the right ventricle or elsewhere in the heart, enabling catheter 162 and balloon 166 (particularly if filled with an ultrasound-opaque fluid) as well as left ventricle 167, cusps 164 and annulus 165 of the mitral valve to be visualized. This visualization enables catheter 162 and balloon 166 of device 160 to be properly positioned in annulus 165. Energy is emitted until the cusps are seen to close properly. Energy is emitted as described for FIG. 10.

Balloon 166 can be deflated and catheter 162 withdrawn into guide catheter 163, moving deflated balloon into ventricle 167. If, using the AcuNav® catheter in color Doppler mode, blood is still seen spurting from the mitral valve during the heart's compression (systole) as red flow into a blue or violet fluid, balloon 166 can again be positioned in annulus 165 and energy may again be emitted. This procedure can be repeated until the leakage of blood is seen to cease, indicating proper closure of cusps 164.

When withdrawn into ventricle 167 and positioned near or in contact with the chordae tendinae 168, apparatus 10 can also be used to shrink chordae tendinae 168, applying energy as referenced for FIG. 10.

If desired, energy is emitted only during diastole, when the heart's electrical activity is minimal, to avoid the risk of an arrhythmia (uncontrolled heartbeat). This is accomplished by synchronizing one-half second or shorter pulses of energy with the patient's electrocardiogram (ECG), after an appropriate delay time from the “r” wave of the ECG, as more fully described in U.S. Pat. No. 6,224,566 to Loeb et al, which is assigned to a subsidiary of the owner hereof, and co-owned U.S. Pat. No. 4,788,975 to Shturman et al. both of which are incorporated herein by reference to the extent not inconsistent.

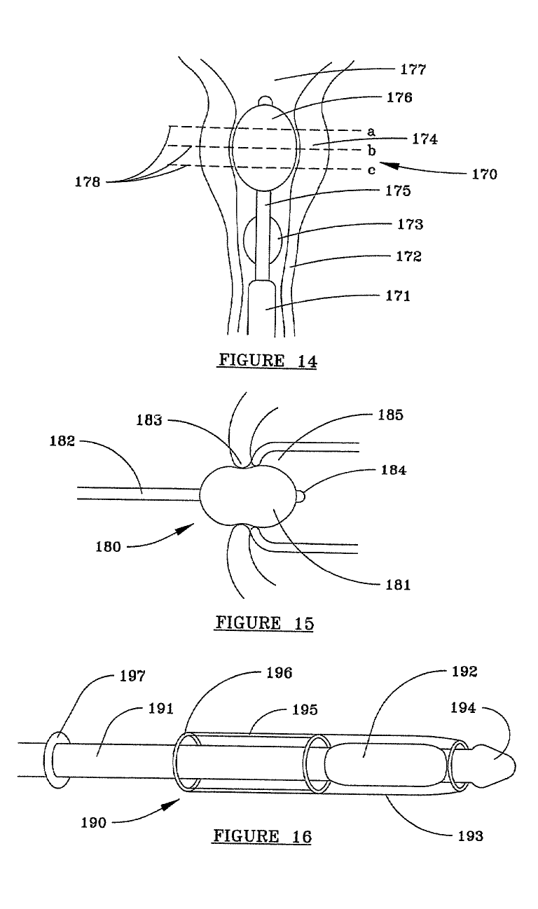

In FIG. 14, the distal end portion 170 of apparatus 10 of FIG. 1 is seen inserted through a channel (not separately shown) of endoscope 171, which has been positioned in male urethra 172 proximal to veru montaneum 173, a raised portion of the floor of urethra 172 just proximal to lobes 174 of the prostate gland. Catheter 175 has been extended out of endoscope 171, distal to veru montaneum 173, until balloon 176, which in this embodiment is made of a non-compliant material, is positioned between lobes 174 of the prostate. When properly positioned, balloon 176 is inflated between lobes 174 of the prostate, just beneath bladder 177.

To shrink, coagulate and cause scarring in the lobes of the prostate, light, microwave or ultrasound energy of the type, amount, pattern, positions and duration described for FIG. 10 above, is emitted radially in a 360° arc through balloon 176, while a cooled fluid is circulated through balloon 176 to avoid damage to the urethra either before the emission of energy to pre-cool the tissue immediately underlying the urethra and during the emission of energy, or only during the emission of energy.

Preferably, light or focused ultrasound, microwave or other electromagnetic energy of the type, amount and duration described in reference to FIG. 10, is emitted laterally, at an angle of about 90° to 110° from the axis of catheter 175, in an arc of about 90° through balloon 176 at, for example, the 2, 4, 8 and 10 o'clock positions to avoid emitting an excessive amount of energy at the 6 and 12 o'clock positions, as other delicate tissues underlie the urethra at these positions. Alternatively, such energy is emitted in relatively lesser amounts or durations at the 6 and 12 o'clock positions and in relatively greater amounts or durations at the 3 and 9 o'clock positions, or in any other desired pattern. The emission of energy may be repeated at various positions or levels within the prostate, depending on the length of lobes 174 from veru 173 to bladder 177, for example, at one or more of levels a, b and c, as indicated by dotted lines 178. Lateral emission of light energy in the prostate is described in more detail in the aforementioned co-owned U.S. Pat. No. 5,437,660.

While not shown in FIGS. 1, 2 and 5 through 11, the distal end portion of apparatus 10 of FIG. 1 is optionally inserted through a channel of an endoscope, and the balloon device may be positioned under direct or videoscopic vision, as shown in this FIG. 14.

In FIG. 15, an alternate distal end portion configuration is shown for a medical device 180. Medical device 180 includes a balloon 181, which is attached about the distal end portion of catheter 182, and has been positioned and expanded in anus 183. The distal end 184 of device 180 extends only slightly into rectum 185.

While balloon 181 is made of a compliant material, as shown in this embodiment, balloon 181 may also be made of a non-compliant material. When positioned and balloon 181 expanded, the type, amount, patterns, positions and duration of emission of energy is as described in reference to FIG. 10. The emission of energy is employed radially over a 360° arc or laterally, at an angle of about 90° to 110° from the axis of catheter 182 in an arc of about 90° at, for example, the 12, 3, 6 and 9 o'clock positions to shrink the tissue underlying anus 183 to treat fecal incontinence.

Cooled fluid is circulated either both before the emission of energy to pre-cool the tissue underlying the anus and during the emission of energy, or only during the emission of energy.

As seen in FIG. 16, an alternate embodiment of the distal end portion 190 of apparatus 10 of FIG. 1 is shown. Distal end portion 190 includes catheter 191, whose distal end is encased in balloon 192. To protect balloon 192 from damage while device 190 is being inserted into the body, a cylindrical, hollow plastic shield 193, whose distal end 194 has been rounded to minimize damage to tissue, is movably disposed over balloon 192. One or more plastic or metal rods or wires 195 extend proximally from shield 193 and terminate at ring 196. Flange 197 is fixedly attached to catheter 181 proximally from ring 186. If, for example, the length of wires 195 is equal to the length of balloon 192, and if flange 197 is spaced the same distance from ring 196, when ring 196 is manually retracted up to flange 197, shield 193 has been retracted a distance sufficient to fully expose and not confine balloon 192.

In an alternate embodiment, distal end 194 of catheter 191 has a sharp point enabling it to be inserted directly into tissue to reach a desired position. In a most preferred embodiment, as earlier described in reference to FIG. 6, the cooling fluid circulated through the balloon of all of the medical treatment embodiments described herein preferably contains a multiplicity of extremely small albumin microspheres or inert particles which reflect and diffuse the radiant energy, but do not appreciably absorb the energy, making the emission of energy from the balloon more uniform and diffuse, while not increasing the temperature of the cooling fluid.

The most preferred cooling fluid contains fumed silica, commercially available under the designation “Cab-O-Sil” from Cabot Corporation (Boston, Mass.), and comprising microscopic particles of silica which effectively reflect, but do not appreciably absorb, light, ultrasound or microwave energy. In the event of leakage from the balloon, fumed silica particles, do not react biologically with tissues and are removed by macrophages of the body's reticulo-endothelial system. Furthermore, fumed silica particles remain in suspension for extended periods of time. These abilities make fumed silica an exceptionally desirable energy diffusing means for use in the cooling fluid.

Numerous variations and modifications of the embodiments described above may be effected without departing from the spirit and scope of the novel features of the invention. It is to be understood that no limitations with respect to the specific methods or systems illustrated herein are intended or should be inferred. It is, of course, intended to cover by the appended claims all such modifications as fall within the scope of the claims.

What is claimed is:

1. A medical device suitable for radiant-energy treatment of tissue within a mammalian body and adapted for use with a source of cooled fluid, the medical device comprising:
an elongate transmission line having a proximal end portion and a distal end portion, said proximal end portion operably connectable to a source of radiant-energy;
a radiant energy emitter coupled to said distal end portion to receive energy therefrom;
an insertion catheter defining an inlet confined flow passageway in fluid communication with said source of cooled fluid, an outlet confined flow passageway, and a transmission line guideway for receiving said transmission line; and
an energy-transmissive balloon at least partially surrounding said emitter for providing a tissue-contacting coolant chamber, said chamber being in fluid communication with the inlet and outlet confined flow passageways.

2. The device according to claim 1 wherein said balloon is constructed of a substantially compliant polymeric material.

3. The device according to claim 1 wherein said balloon is constructed of a polymeric material adapted to be substantially non-compliant.

4. The device according to claim 1 wherein said balloon is constructed of a material selected from the group consisting of a natural rubber, a polyurethane, a polyethylene, a polyethylene terephthalate, a polyester, a copolyester, a polyvinyl chloride, a copolymer of vinyl chloride, vinylidene chloride, and composites thereof.

5. The device according to claim 1 further comprising a source of intense incoherent light and wherein said transmission line includes an optical fiber optically linked to said source of intense incoherent light.

6. The device according to claim 5 wherein said source of intense incoherent light includes a filtered lamp system.

7. The device according to claim 5 wherein said source of intense incoherent light includes a device selected from the group consisting of a mercury lamp, a tungsten lamp, a xenon lamp, and a tungsten-halogen lamp.

8. The device according to claim 1 further comprising a microwave generator and wherein said transmission line is a coaxial conductive cable and said emitter is a microwave antenna.

9. The device according to claim 1 wherein said emitter is a piezoelectric acoustic emitter.

10. The device according to claim 1 further comprising a laser energy source and wherein said energy transmission line includes an optical fiber optically coupled to said laser energy source.

11. The device according to claim 1 wherein said emitter is adapted to emit radiant energy substantially lateral to the axis of said transmission line.

12. The device according to claim 1 wherein said emitter is adapted to emit radiant energy substantially lateral to the axis of said transmission line in a pattern defining an arc of about 90°.

13. The device according to claim 1 further comprising a source of cooling fluid in fluid communication with said inlet passageway for filling said balloon and cooling said chamber.

14. The device according to claim 1 including a source of cooled fluid in fluid communication with said inlet passageway for filling said balloon and cooling said chamber and wherein said cooled fluid is selected from the group consisting of chilled de-ionized water, chilled saline, and cryogenic gas.

15. The device according to claim 1 further comprising a coolant circulating system in communication with said inlet passageway and said outlet passageway for pressurizing and circulating said cooled fluid through said balloon.

16. The device according to claim 15 including a temperature sensor operably linked to said coolant circulating system and positioned to measure the temperature of said fluid.

17. The device according to claim 15 including a pressure sensor operably linked to said coolant circulating system and positioned to measure the pressure of said cooled fluid.

18. The device according to claim 15 in which said circulating system includes a refrigeration unit for cooling said cooled fluid.

19. The device according to claim 15 wherein the circulator is selected from the group consisting of a peristaltic pump, a diaphragm pump, a piston pump, a bellows pump, a syringe pump, a roller and ball pump.

20. The device according to claim 1 further comprising a fluid coolant containing microscopic, radiant-energy dispersing particles that do not appreciably absorb said radiant energy.

21. The device according to claim 1 further comprising a source of fluid coolant including wave-energy dispersing particles selected from the group consisting of microscopic albumin microspheres, quartz and fumed silica.

22. The device according to claim 1 further comprising a source of fluid coolant including fused silica particles having a diameter of less than about 50 microns.

23. The device according to claim 1 further comprising a source of fluid coolant including fused silica particles present in the range of about 2.5 to about 25 percent, based on the total weight of the coolant.

24. The device according to claim 1 wherein said emitter is adapted to emit radiant energy substantially lateral to the axis of said transmission line in a pattern defining an arc of 360°.

25. A medical device suitable for radiant-energy treatment of tissue within a mammalian body and adapted for use with a source of cooled fluid, the medical device comprising:
an elongate transmission line having a proximal end portion and a distal end portion, said proximal end portion operably connectable to a source of radiant-energy;
a radiant energy emitter coupled to said distal end portion to receive energy therefrom;
an insertion catheter defining an inlet confined flow passageway in fluid communication with said source of cooled fluid and a transmission line guideway for receiving said transmission line;
an energy-transmissive balloon at least partially surrounding said emitter for providing a tissue-contacting coolant chamber, said chamber being in fluid communication with the inlet and outlet confined flow passageways; and
a regulator valve in said balloon for allowing cooled fluid to exit said coolant chamber when said chamber reaches a predetermined fluid pressure.

26. A medical device suitable for radiant-energy treatment of animal tissue comprising:
an elongate optical conduit having a proximal end region and a distal end region extending along a longitudinal axis and terminating in an energy delivery, distal end defined thereon for emitting laser radiation transmitted by said conduit, said proximal end region being connectable to a laser energy source;
a beam splitting lateral emitter mounted on said distal end region and operably associated with said distal end for directing laser energy to said tissue;
an insertion catheter defining an inlet confined flow passageway, an outlet confined flow passageway, and a transmission line guideway for receiving said conduit; and
a laser-transmissive balloon at least partially surrounding said emitter for providing a tissue-contacting coolant chamber, said chamber being in fluid communication with the inlet and outlet confined flow passageways.

27. The device according to claim 26 wherein said lateral emitter further comprises:
an element defining a cavity within which said distal end of said conduit is received, the cavity having a distal end wall for blocking transmission of a laser energy beam coaxial with said distal end of said conduit;
a laterally open aperture to said cavity, said aperture being open to fluid communication from outside said element through said aperture into said cavity; and
a beam splitter, disposed within said cavity, for receiving laser energy transmitted through said conduit and for directing at least a first portion of said received laser energy as a laser energy beam exiting said element along a lateral beam path through said aperture.

28. The device according to claim 26 wherein said beam splitter includes a reflecting surface defined on said closed distal end wall and arranged to reflect said first portion of said received laser energy.

29. The device according to claim 26 wherein said lateral emitter further comprises:
a reflector positioned generally axially aligned with said energy delivery distal end for reflecting said emitted radiation in a beam radiating substantially transversely of, and substantially around, said axis;
a housing defining a central bore in which said conduit is disposed, with the distal end of said conduit projecting beyond said bore, said housing including a laser transmissive sleeve having opposed first and second ends, said first end being mounted to a distal end of said catheter; and
an atraumatic plug positioned at said sleeve's second end.

30. The device according to claim 29 wherein said balloon has a first waist sealed to said catheter and a second waist sealed to an atraumatic plug mounted to said emitter.

31. The device according to claim 29 wherein said reflector includes a generally conical surface defined around a central axis that is coincident with said longitudinal axis of said conduit distal end region.

32. The device according to claim 29 in which said reflector includes a metallic coating defining a reflecting surface.

33. A medical device suitable for radiant-energy treatment of animal tissue from a radiant-energy source, the device comprising:
an insertion catheter having a proximal end and a distal end, said catheter defining a coolant inlet confined flow passageway, a coolant outlet confined flow passageway, and a conduit guide;
a wave-energy transmission cable slidably received in said conduit guide and having a distal end portion extending past said distal end of said catheter, and having a proximal end portion adapted to be operably coupled to said radiant energy source;
a radiant energy emitter head mounted to said distal end portion; and
a transparent balloon sealed at one end about said distal end of said catheter and sealed at the other end about an end cap, said catheter, said balloon, and said cap defining an inflatable coolant chamber substantially surrounding said emitter head and in fluid communication with both said inlet and said outlet confined flow passageways.

34. The device according to claim 33 further comprising a source of fluid coolant containing microscopic particles for scattering light through the interior of said balloon which do not appreciably absorb said radiant energy.

35. A method for shrinking, coagulating or scarring tissue surrounding a duct, hollow organ or body cavity of a person having a medical condition requiring treatment, comprising the steps of:
positioning an expandable coolant balloon adjacent the tissue to be treated, the coolant balloon being carried by the distal end of an energy-emitting catheter having a wave energy emitter at least partially surrounded by said balloon;
circulating fluid coolant through said coolant balloon to expand said coolant balloon and cool said tissue; and
energizing said emitter at a predetermined power level to emit radiant energy so as to irradiate the tissue to produce a zone of shrinkage in the irradiated tissue, while continuing to circulate fluid coolant.

36. The method of claim 35 further comprising controlling the pressure of said coolant fluid by adjusting the rate of fluid coolant infusion, while energizing said emitter.

37. The method of claim 35 further comprising controlling the temperature of said coolant fluid by adjustably cooling said fluid coolant while energizing said emitter.

38. The method of claim 35 wherein said radiant energy is obtained from a laser.

39. The method of claim 35 wherein said radiant energy is obtained from a microwave generator.

40. The method of claim 35 wherein said radiant energy is obtained from a high intensity white light generator.

41. The method of claim 35 wherein said radiant energy is obtained from an ultrasonic emitter.

42. The method of claim 35 wherein fluid coolant is circulated through said coolant balloon for a time period sufficient to cool said tissue before energizing said emitter.

43. The method of claim 35 wherein said fluid coolant contains a multiplicity of microscopic particles which diffuse the radiant energy without appreciable absorption of said energy.

44. The method of claim 35 wherein the medical condition requiring treatment is selected from the group consisting of gastroesophageal reflux disease, female stress incontinence, fecal incontinence, vesico-uretal reflux, an incompetent heart valve, and benign prostatic hyperplasia.

45. A method for shrinking, coagulating or causing scarring of tissue adjacent a duct, hollow organ or body cavity of a patient having a condition requiring treatment, comprising the steps of:
positioning a radiant-energy transmissive, expandable coolant balloon adjacent the tissue to be treated, the coolant balloon being carried by the distal end of a laser catheter having a lateral-lasing emitter at least partially surrounded by said balloon;
circulating fluid coolant through said coolant balloon to expand and press said coolant balloon against said tissue;
energizing the lateral-lasing emitter at a predetermined power level to emit laser energy in a direction substantially transversely to the longitudinal axis of the laser catheter so as to irradiate the tissue to be treated for a predetermined time period to produce a zone of shrinkage in the irradiated tissue.

46. The method of claim 45 wherein fluid coolant is circulated through said coolant balloon for a time period sufficient to cool said tissue before energizing said emitter.
